(19) United States
(12) Patent Application Publication
(10) Pub. No.: US 2020/0108009 A1
(43) Pub. Date: Apr. 9, 2020

(54) BIOAVAILABLE AEROSOLIZED SUPPLEMENT FORMULATIONS AND METHODS THEREOF

(71) Applicant: Inhale Health LLC, Beverly Hills, CA (US)

(72) Inventors: Mario Danek, Los Angeles, CA (US); Joseph G. Walsh, Los Angeles, CA (US); Daniel Frederick Woolf Shapiro, Los Angeles, CA (US)

(21) Appl. No.: 16/617,460

(22) PCT Filed: May 25, 2018
(86) PCT No.: PCT/US18/34762

§ 371 Date: Nov. 26, 2019

Related U.S. Application Data
(63) Continuation of application No. 15/606,546, filed on May 26, 2017.

(51) Int. Cl. A61K 9/00; A61K 31/197; A61K 31/375; A61K 31/4045; A61K 31/4188; A61K 31/4415; A61K 31/465; A61K 31/51; A61K 31/522; A61K 31/525; A61K 31/714; A61K 47/10

(52) U.S. Cl. CPC A61K 9/007; A61K 31/197; A61K 31/375; A61K 31/4045; A61K 31/4188; A61K 31/4415; A61M 11/042; A61K 31/51; A61K 31/522; A61K 31/525; A61K 31/714; A61K 47/10; A61K 31/465

(57) ABSTRACT

Methods of preparing inhalable supplements are disclosed. The methods involve aerosolizing a liquid comprising a carrier and a supplement by heating the liquid to a temperature that retains the bioavailability of the supplement. Also disclosed are methods and kits for treating a subject with an inhalable supplement.

### CROSS-REFERENCE TO RELATED APPLICATIONS

This application claims the benefit of U.S. patent application Ser. No. 15/606,546, filed May 26, 2017, which is incorporated herein by reference in its entirety.

### BACKGROUND OF THE INVENTION

### Field of the Invention

The present disclosure provides formulations of bioavailable aerosolized supplements and methods for use. More particularly, the present disclosure relates to methods of preparing inhalable supplements, to related supplement preparations, and to methods and kits for treating a subject with an inhalable supplement.

### Description of the Related Art

Dietary supplements provide supplemental nutrition and health benefits to subjects in need. Various vitamins, for example, are essential nutrients that contribute to a subject's health. Vitamin B12 (cobalamin), for example, contributes to proper red blood cell formation, neurological function and DNA synthesis. Vitamin B6 (pyridoxine) is involved in neurotransmitter biosynthesis, maintaining normal levels of homocysteine and maintaining immune function. In addition, vitamin B1 (thiamine) is involved in energy metabolism and the growth, development and function of cells. Further, vitamin B2 (riboflavin) is an essential component of the coenzymes flavin mononucleotide and flavin adenine dinucleotide (FAD), which are involved in energy production, cellular function, growth, and development, and metabolism of fats, drugs, and steroids. Consumption of supplements such as vitamins in pill format, however, can be highly inefficient due to low oral absorption rates. Administration of supplements through injection is known to be effective, but inefficient and undesirable due to its invasive nature. Poor absorption rates of supplements and nutrients through digestive inefficiencies are well known (Meyer, L M, et al. Am. J. Med. Sci., 1950, 220:604; Conley, C M, et al. J. Lab. &Clin. Med., 1951, 38:84-95). Previous studies have demonstrated that oral and sublingual administration of vitamin B12, for example, result in unsatisfactory clinical and hematologic responses due to poor absorption through digestive inefficiencies (Meyer, L M, et al. 1950; Conley, C M, et al. 1951; Ungley, C C. Brit. Med. J., 1950, 2:905). Studies have shown that digestive inefficiencies result in only about 10 mcg of a 500 mcg (or 2 percent) of an oral supplement being absorbed in healthy individuals (National Institutes of Health, Office of Dietary Supplements Fact Sheet, updated and accessed Mar. 2, 2018; Carmel, R. Blood, 2008, 112:2214-2221). Gastric absorption rates are even less efficient in individuals with gastrointestingal disorders, such as celiac and Crohn's disease, those with pernicious anemia, elderly individuals (particularly those with atrophic gastritis), and individuals who have had gastrointestinal surgery (Institute of Medicine, Washington, D.C.: National Academy Press, 1998; Johnson, M A. Nutr Rev, 2007, 65:451-458; Andres E, et al. J Fam Pract, 2007, 56:537-542; Carmel, R. Baillieres Clin Haematol, 1995, 8:639-655). Conversely, administration of, for example, vitamin B12, through inhalation has been shown to increase effectiveness of absorption by what is believed to be direct diffusion through the respiratory mucosa into the lung alveoli and capillaries; this has been shown to be true in normal subjects, subjects with pulmonary diffusion defects and subjects with vitamin B12 deficiency (Shinton, N K and Singii, A K. Brit. J. Haemat., 1967, 13:75). However, there remains a need for providing nutritional supplements to subjects in a more efficient and effective manner.

### SUMMARY OF THE INVENTION

The inventors have identified that the invention disclosed herein provides a non-invasive method of administering supplements that bypasses the above-identified inefficiencies by utilizing direct absorption of an inhaled, aerosolized supplement through diffusion to the lungs, according to the disclosure. Although this invention as disclosed herein is not limited to specific advantages or functionality, in one aspect the present disclosure provides a method of preparing an inhalable supplement comprising aerosolizing a liquid comprising a carrier and an inhalable supplement by heating the liquid to a temperature of about 25° C. to about 300° C. using an atomizer, thereby providing an inhalable vapor containing the supplement. In some embodiments, the atomizer further comprises a heating element connected to a battery, wherein the power generated by the battery connected to the heating element is about 0.2 to about 200 Joules/second, and wherein the liquid is heated for 1 to 10 seconds.

In another aspect the present disclosure provides a method of treating a subject with a supplement comprising administering to the subject a therapeutically effective amount of an inhalable vapor containing the supplement.

In another aspect, the present disclosure provides a kit for treating a subject with a supplement comprising: a. a therapeutically effective amount of an inhalable vapor containing the supplement; b. an apparatus for administering said compound; and c. instructions for use.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the methods and compositions of the disclosure, and are incorporated in and constitute a part of this specification. The drawings illustrate one or more embodiment(s) of the disclosure, and together with the description serve to explain the principles and operation of the disclosure.

FIG. 1 illustrates that melatonin is present in the aerosolized state. (A) Chromatogram of sample of product containing melatonin prior to vaporization, tested by HPLC. (B) Chromatogram of same sample after vaporization into water reservoir present in the impinger, tested by HPLC. (HPLC Instrument method: C18 pH6 full gradient; Run Time: 15 min; Injection volume: 5 μl; Wavelength: 210.0 nm; Bandwidth: 10; Dilution Factor: 1.0000; Sample weight: 1.0000).

FIG. 2 illustrates that caffeine is present in the aerosolized state. (A) Chromatogram of sample of product containing caffeine prior to vaporization, tested by HPLC. (B) Chromatogram of same sample after vaporization into water reservoir present in the impinger, tested by HPLC. (HPLC Instrument method: C18 pH6 full gradient; Run Time: 15 min; Injection volume: 5 μl; Wavelength: 210.0 nm; Bandwidth: 10; Dilution Factor: 1.0000; Sample weight: 1.0000).

FIG. 3 illustrates that L-theanine is present in the aerosolized state. (A) Chromatogram of sample of product containing L-theanine prior to vaporization, tested by HPLC. (B) Chromatogram of same sample after vaporization into water reservoir present in the impinger, tested by HPLC. (HPLC—Instrument method: HILIC H3PO4 pH6 CAN isocratic; Run Time: 10 min; Injection volume: 5 μl; Wavelength: 210.0 nm; Bandwidth: 10; Dilution Factor: 1.0000; Sample weight: 1.0000).

FIG. 4 illustrates that B vitamins are present in the aerosolized state. (A) Chromatogram of control sample containing vitamins B1, B2, B6 and B12 prior to vaporization, tested by HPLC through appropriate protocols to identify the presence of each of the vitamins. (B) Chromatogram of same sample after vaporization into water reservoir present in the impinger, tested by HPLC.

FIG. 5 illustrates an increase in Vitamin B12 blood serum levels from in vivo studies. Three doses (60 inhalations/dose) of vitamin B12 product (20 mg/ml) taken in 24 hour intervals; serum vitamin B12 levels measured at time zero, prior to initial dose and 2 hours after inhalation of first and third doses.

### DETAILED DESCRIPTION OF THE INVENTION

Before the disclosed processes and materials are described, it is to be understood that the aspects described herein are not limited to specific embodiments or configurations, and as such can, of course, vary. It is also to be understood that the terminology used herein is for the purpose of describing particular aspects only and, unless specifically defined herein, is not intended to be limiting.

It is also to be understood that unless clearly indicated otherwise by the context, embodiments disclosed for one aspect or embodiment of the invention can be used in other aspects or embodiments of the invention as well, and/or in combination with embodiments disclosed in the same or other aspects of the invention. Thus, the disclosure is intended to include, and the invention includes, such combinations, even where such combinations have not been explicitly delineated.

### Definitions

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the term “contacting” includes the physical contact of at least one substance to another substance.

As used herein, the terms “vaporize” or “vaporized” and “aerosolize” or “aerosolized” are used interchangeably herein and refer to heating a liquid composition to diffuse or suspend the composition in the air, such that it is suitable for inhalation into a subject's lungs.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like.

As used herein, the term “disease” refers to any condition that is abnormal, such as a disorder or a structure or function that affects part or all of a subject.

As used herein, the term “supplements” or “nutritional compositions” are used interchangeably herein and refer to target compositions that are able to be solubilized and are suitable for atomization. By way of non-limiting example, supplements and nutritional compositions include vitamins, hormones, antioxidants, stimulants, phytonutrients, phytochemicals and amino acids.

As used herein, the term “electronic inhaler” or “e-inhaler” are used interchangeably herein and refer to a device capable of vaporizing a liquid composition for inhalation, and which includes an atomizer and a battery. By way of non-limiting example, an electronic inhaler can include e-cigarette-type devices.

Bioavailability of Inhaled Aerosolized Compositions

Inhalation is an emerging method to absorb target compositions, including but not limited to supplements, such as amino acids, hormones, antioxidants, stimulants, phytochemicals, phytonutrients and vitamins directly into the bloodstream. The disclosure herein provides non-invasive methods of administering supplements that bypasses the inefficiencies of the digestive system by utilizing direct absorption of an inhaled, aerosolized supplement through diffusion to the capillaries via the lung alveoli. As a non-limiting example, vitamin sizes range from angstroms to a few nanometers; accordingly, aerosols generated by vaporizers can administer large doses of target compositions, including vitamins and essential nutrients.

The Clearance Mechanisms of the Lung

The lung engages mechanisms, including airway geometry, humidity and clearance mechanisms, to filter unwanted airborne particles and prevent them from entering the body (Labiris, N R & Dolovich, M B. J Clin Pharmacol, 2003, 56:588-599). Progressive branching and narrowing of the airways promote impaction of particles. In addition, the lung has a relative humidity of approximately 99.5% (Labiris & Dolovich, 2003).

Particle size is an important variable to determine the dose deposited and the distribution of aerosolized compositions in the lung (Labiris & Dolovich, 2003). Most therapeutic aerosols are heterodisperse, consisting of a wide range of particle sizes and described by the log-normal distribution, with the log of the particle diameters plotted against particle number, surface area or volume (mass) on a linear or probability scale and expressed as absolute values or cumulative percentage (Labiris & Dolovich, 2003).

Depending on their size, particles can be deposited by inertial impaction, gravitational sedimentation or diffusion (Brownian motion). Particles not deposited during inhalation are exhaled. Particles smaller than 0.5 mm are mainly deposited by diffusion. Drug particles are known to be hygroscopic and grow or shrink in size in high humidity, such as in the lung. A hygroscopic aerosol that is delivered at relatively low temperature and humidity into one of high humidity and temperature would be expected to increase in size when inhaled into the lung. The rate of growth is a function of the initial diameter of the particle, with the potential for the diameter of fine particles <1 mm to increase five-fold compared with two-to-three-fold for particles >2 mm (Labiris & Dolovich, 2003).

To take advantage of this direct delivery mechanism to the lungs, aerosolized droplets smaller than 500 nanometers in diameter avoid sedimentation in airways leading to deposition by diffusion in the alveoli (Grana, R, et al. Circulation, 2014, 129(19):1972-1986). As the major component of the aerosol, the kinetic and thermodynamic behavior of vegetable glycerin dictates the behavior of the mixture. Initial size of vegetable glycerin aerosols have a peak diameter of 120-180 nanometers (Grana, R, et al., 2014; Zhang, Y, et al. Nicotine & Tobacco Research, 2012, 15(2):501-508). The distribution of particle diameters for aerosols are comparable cigarette smoke, which falls within the parameters for deposition by diffusion, providing optimal alveolar deposition (Zhang, Y, et al, 2012).

After deposition in the alveoli, absorption by diffusion into the bloodstream is the major method of clearance from the lungs (Labiris & Dolovich, 2003). Molecules <6 nm rapidly diffuse into the bloodstream following inhalation (Labiris & Dolovich, 2003). Up to 18% of the aerosol volume is deposited in the alveoli and up to 27% of the total aerosol volume is delivered for systemic circulation (Grana, R, et al., 2014). This is in line with the 25% to 35% total absorption rate of cigarette smoke, a proven delivery mechanism (Grana, R, et al., 2014). Inhalation can absorb vitamins over 10 times more efficiently than pill formats with faster delivery to the bloodstream. Inhalation enables you to reap your desired benefits immediately.

In view of the present disclosure, the methods and compositions described herein can be configured by the person of ordinary skill in the art to meet the desired need. In general, the disclosed materials provide formulations of inhalable supplements and methods of preparing. The methods comprise aerosolizing a liquid comprising a carrier and an inhalable supplement by heating the liquid using an atomizer, thereby providing an inhalable vapor containing the inhalable supplement. The disclosed methods advantageously aerosolize the inhalable supplement so as to provide a bioavailable supplement having a high pulmonary absorption rate.

In another aspect, the present disclosure provides for methods of treating a subject with a supplement comprising administering to the subject a therapeutically effective amount of an inhalable vapor containing a supplement. In some embodiments, the inhalable vapor comprises a liquid comprising a carrier and a supplement, and wherein the liquid is aerosolized by heating the liquid to a temperature of about 25° C. to about 300° C. using an atomizer. In some embodiments, the inhalable vapor is administered in a single dose or in multiple doses.

In another aspect, the present disclosure provides for kits for treating a subject with an inhalable supplement.

In some embodiments, the liquid comprising the carrier and the inhalable supplement is present as a mixture, for example, a homogeneous mixture or a heterogeneous mixture. In some embodiments, the liquid comprising the carrier and the inhalable supplement is present as a suspension. In some embodiments, the liquid comprising the carrier and the inhalable supplement is present as a solution. In some embodiments, the liquid is free of nicotine. In some embodiments, the liquid comprises nicotine.

In another aspect, the liquid comprises about 51% (w/w) to about 99.99999% (w/w) carrier. In some embodiments, the liquid comprises about 0.00001% (w/w) to about 49% (w/w) inhalable supplement.

There are health concerns of toxic carbonyl exposure when heat is used to aerosolize suspensions of propylene glycol and vegetable glycerin [1]. When excess heat is applied to these solvents, volatile carbonyl degradation products are known to form at increasing rates (e.g. formaldehyde, acetaldehyde and acrolein) (Grana, R, et al., 2014). Propylene glycol has been excluded from all products as it is known to oxidize and degrade at low temperatures. Products instead utilize carriers comprising, for example, organic vegetable glycerin suspensions to aerosolize our inhalable nutrients. Five-watt heating coils have similar technical specifications and can serve as an effective model for our products. During intermittent use of a 5 W coil, temperatures remain well below 200° C., which has been shown to avoid substantial increase in carbonyl production (Wang, P, et al. PLoS ONE, 2017, 12(1): e0169811).

Suitable carriers include, but are not limited to glycerin, such as vegetable glycerin, 1,2-propanediol, 1,3-propanediol, and combinations thereof. In some embodiments, the carrier comprises a mixture of glycerin, 1,2-propanediol, and 1,3-propanediol. In some embodiments, the carrier comprises a mixture of glycerin and 1,3-propanediol. In some embodiments, the carrier comprises a mixture of glycerin and 1,2-propanediol. In some embodiments, the carrier comprises a mixture of 1,2-propanediol and 1,3-propanediol. In some embodiments, the carrier comprises glycerin and 1,3-propanediol present in a weight ratio of 1:1000 to 1000:1. In some embodiments, the carrier comprises glycerin and 1,2-propanediol present in a weight ratio of 1:1000 to 1000:1.

Suitable supplements include, but are not limited to, vitamin B1, vitamin B2, vitamin B6, vitamin B12, ascorbic acid, biotin, melatonin, L-theanine, caffeine, and combinations thereof. In some embodiments, the inhalable supplement comprises vitamin B1, vitamin B2, vitamin B6, vitamin B12, ascorbic acid, biotin, or combinations thereof. In an embodiment, the inhalable supplement comprises vitamin B12. In some embodiments, the inhalable supplement comprises a mixture of vitamin B1, vitamin B2, vitamin B6, and vitamin B12. In some embodiments, the inhalable supplement comprises ascorbic acid. In some embodiments, the inhalable supplement comprises biotin. In some embodiments, the inhalable supplement comprises melatonin. In some embodiments, the inhalable supplement comprises L-theanine. In some embodiments, the inhalable supplement comprises caffeine. In some embodiments, the inhalable supplement comprises a mixture of L-theanine and caffeine.

In another aspect, the inhalable supplement has a boiling point greater than the temperature to which the liquid is heated.

In another aspect, the liquid is heated for about 1 to about 10 seconds.

In another aspect, the atomizer comprises a heating element to vaporize the liquid. In some embodiments, the atomizer further comprises a delivery mechanism that actively or passively transports the liquid to the heating element. In some embodiments, the atomizer further comprises a wicking material as a delivery mechanism to draw the liquid to the heating element. In some embodiments, the atomizer does not contain a wicking material. In some embodiments, the liquid is dripped directly onto the heating element. In some embodiments, the atomizer is a ceramic atomizer. In some embodiments, the heating element has a resistance of about 0.4 ohms to about 6 ohms. In some embodiments, the heating element is connected to a 1- to 9.1-volt battery.

In another aspect, the vapor produced by the method described herein contains particles having a mass mean aerodynamic diameter of less than about 50 microns.

In another aspect, inhalation of the vapor by a subject increases plasma levels of the supplement in the subject. In some embodiments, plasma levels of the supplement are detectably increased compared to plasma levels before inhalation of the vapor within about 1 to 120 minutes, for example, about 30 to about 120 minutes or about 30 to about 90 minutes, or about 30 to about 60 minutes, or about 30 to about 45 minutes, or about 60 to about 120 minutes, or about 60 to about 90 minutes, or about 60 to about 75 minutes after inhalation of the vapor. In some embodiments, blood plasma levels of the supplement in the subject are increased compared to plasma levels before inhalation of the vapor by, for example, at least 3%.

Formulations

The liquid preparations disclosed herein include a carrier component and an inhalable supplement component. The liquid preparations are present in a device that is suitable to aerosolize the liquid, thereby providing an inhalable vapor containing the supplement. Typically, about 0.1 to about 100 ml of liquid is present in the device, for example, about 0.1 to about 10 ml, about 0.5 to about 2 ml, about 1 to 1.5 ml, and/or about 1 to about 1.2 ml. In some cases, about 0.1 mg to about 100 g of liquid is present in the device, for example, about 500 mg to about 1.5 g, about 200 mg to about 1.2 g, about 0.1 mg to about 15 mg, about 0.2 mg to about 10 mg, about 0.5 mg to about 5 mg, about 0.5 mg to about 3 mg, about 1 mg to about 2 mg, about 1.1 mg to about 1.7 mg, and/or about 1.3 mg to about 1.5 mg of liquid. The liquid comprising the carrier and the inhalable supplement can be present as a mixture, for example, a homogeneous mixture or a heterogeneous mixture, a solution, or a suspension.

The carrier component includes any carrier suitable for aerosolizing the inhalable supplement at relatively low temperatures, such as at a temperature below the boiling point of the inhalable supplement. Without wishing to be bound by theory, it is believed that aerosolizing the inhalable supplement at a temperature below its boiling point reduces degradation of the inhalable supplement, thereby facilitating delivery of the bioavailable inhalable supplement to a subject in an amount sufficient to achieve a desired effect in the subject. The liquid preparations contain an amount of carrier suitable to allow a desired amount of inhalable supplement to be aerosolized under conditions that allow the bioactive supplement to be delivered to a subject. In some embodiments, the liquid contains the carrier component and the inhalable supplement in a weight ratio of about 1:1 to about 1000:1, about 2:1 to about 1000:1, about 5:1 to about 1000:1, about 10:1 to about 1000:1, about 5:1 to about 10:1, about 7:1 to about 10:1, and/or about 1:1 to about 1:1000. Suitable carriers include, but are not limited to glycerin, such as vegetable glycerin, 1,2-propanediol, 1,3-propanediol, and combinations thereof. In some cases, the carrier is a mixture of glycerin, 1,2-propanediol, and 1,3-propanediol. In some embodiments, the carrier is a mixture of glycerin and 1,3-propanediol. In some embodiments, the carrier is a mixture of glycerin and 1,2-propanediol. In some embodiments, the carrier is a mixture of 1,2-propanediol and 1,3-propanediol. In some embodiments, the carrier comprises glycerin and 1,3-propanediol in a weight ratio of 1:1000 to 1000:1, for example, 1:10 to 10:1, 1:7.5 to 7.5:1, 1:5 to 5:1, 1:4 to 4:1, 1:3 to 3:1, 1:2 to 2:1, 1:1.5 to 1.5:1, and/or 1:1. In some embodiments, the carrier comprises glycerin and 1,2-propanediol in a weight ratio of 1:1000 to 1000:1, for example, 1:10 to 10:1, 1:7.5 to 7.5:1, 1:5 to 5:1, 1:4 to 4:1, 1:3 to 3:1, 1:2 to 2:1, 1:1.5 to 1.5:1, and/or 1:1.

The carrier component often comprises more than half of the total weight of the liquid. In some cases, for example, the liquid comprises about 51% (w/w) to about 99.99999% (w/w), about 60% (w/w) to about 99.9999%, about 60% (w/w) to about 99.999%, about 60% (w/w) to about 99.99%, about 60% (w/w) to about 99.9%, about 60% (w/w) to about 95% (w/w), about 70% (w/w) to about 95% (w/w), about 80% (w/w) to about 99.9% (w/w), about 80% (w/w) to about 95% (w/w), about 80% (w/w) to about 90% (w/w), and/or about 85% (w/w) to about 90% (w/w) carrier based on the total weight of the liquid. In such embodiments, the supplement comprises less than half of the total weight of the liquid. In some embodiments, for example, the liquid comprises about 0.00001% (w/w) to about 49% (w/w), about 0.0001% (w/w) to about 40% (w/w), about 0.001% (w/w) to about 40% (w/w), about 0.01% (w/w) to about 40% (w/w), about 0.1% (w/w) to about 40% (w/w), about 5% (w/w) to about 40% (w/w), about 5% (w/w) to about 30% (w/w), about 0.01% (w/w) to about 20% (w/w), about 5% (w/w) to about 20% (w/w), about 10% (w/w) to about 20% (w/w), and/or about 10% (w/w) to about 15% (w/w) supplement based on the total weight of the liquid.

In some embodiments, about 0.1 to about 100 ml of carrier is present in the device, for example, about 0.1 to about 10 ml, about 0.5 to about 2 ml, about 1 to 1.5 ml, and/or about 1 to about 1.2 ml of carrier. In some embodiments, about 0.1 mg to about 100 g of carrier is present in the device, for example, about 500 mg to about 1.5 g, about 200 mg to about 1.2 g, about 0.1 mg to about 15 mg, about 0.2 mg to about 10 mg, about 0.5 mg to about 5 mg, about 0.5 mg to about 3 mg, about 1 mg to about 2 mg, about 1.1 mg to about 1.7 mg, and/or about 1.3 mg to about 1.5 mg of carrier.

In some embodiments, about 0.01 mg to about 100 mg of supplement is present in the device, for example, about 0.03 mg to about 1.5 mg, about 0.05 mg to about 1 mg, about 0.1 mg to about 0.5 mg, about 0.15 mg to about 0.2 mg, about 11 mg to about 50 mg, about 43 mg to about 90 mg, about 37 mg to about 100 mg, and/or about 1 mg to about 17 mg of supplement.

Inhalable supplements for use in the formulations and methods disclosed herein include ingredients with biological activity and/or a biological response. In some aspects, the inhalable supplements provide a benefit to the health of the subject to which the inhalable supplements are administered. Inhalable supplements include, but are not limited to, dietary supplements, stimulants, relaxants, minerals, vitamins, hormones, and other compounds with biological functionality or a biological response within in the subject. Inhalable supplements include, but are not limited to, nutrients, such as vitamins, minerals, amino acids and proteins, fiber, and fatty acids, and biologically active compounds. Suitable supplements include, but are not limited to, vitamin B1, vitamin B2, vitamin B6, vitamin B12, ascorbic acid, biotin, melatonin, L-theanine, caffeine, and mixtures thereof. In some embodiments, the supplement comprises a mixture of vitamin B1, vitamin B2, vitamin B6, and vitamin B12. In some embodiments, the supplement comprises a mixture of L-theanine and caffeine. In some embodiments, the supplement comprises ascorbic acid. In some embodiments, the supplement comprises biotin. In some embodiments, the supplement comprises melatonin. In some embodiments, the supplement excludes nicotine. Further, in some embodiments, the liquid is free of nicotine. In some embodiments, the supplement has a boiling point greater than the temperature to which the liquid is heated. For example, the difference between the temperature to which the liquid is heated and the boiling point of the supplement typically is at least 10° C., for example, at least 20° C., at least 30° C., at least 50° C., at least 75° C., at least 100° C., at least 150° C., and/or at least 200° C.

The liquid formulations may include additional components such as flavorings, crowding reagents, and/or a compound that alters the bioavailability and/or solubility of the inhalable supplement. Suitable flavorings include, but are not limited to French vanilla flavor, cream flavor, regular vanilla flavor, honey, raspberry, strawberry, watermelon, blueberry, peach, cherry, chocolate, dark chocolate, milk chocolate, passion fruit, pomegranate, loganberry, banana, cinnamon, licorice, lychee, dragon fruit, tangerine, banana, tea, milk, lavender, orange, coffee, chamomile, mint, peppermint, spearmint, menthol, cereal, and mixtures thereof.

Methods for Vapor Preparation

The disclosure provides methods for preparing an inhalable vapor containing a supplement. The inhalable vapor is prepared by aerosolizing a liquid preparation containing a supplement as described herein. The liquid preparations are present in a device that is suitable to aerosolize the liquid, thereby providing an inhalable vapor containing the supplement. In particular, the liquid is aerosolized by heating the liquid to a relatively low temperature, such as a temperature below the boiling point of the inhalable supplement. As discussed above, aerosolizing the supplement at a relatively low temperature (i.e., a temperature below the supplement's boiling point) is reduces degradation of the supplement, thereby facilitating delivery of bioactive supplement to a subject. Typically, the liquid is heated to a temperature of about 25° C. to about 300° C., for example, about 25° C. to about 75° C., about 30° C. to about 70° C., about 35° C. to about 105° C., about 100° C. to about 200° C., about 110° C. to about 260° C., and/or about 80° C. to about 300° C.

The liquid is heated for a relatively short period of time (e.g., less than 10 seconds) to avoid overheating of the liquid and to rapidly aerosolize the supplement for delivery to the subject. In some embodiments, the liquid is heated for about 1 to about 10 seconds, for example, about 1 to about 8 seconds, about 1 to about 6 seconds, about 1 to about 5 seconds, about 1 to about 4 seconds, about 1 to about 3 seconds, about 1 to about 2 seconds, about 2 to about 10 seconds, about 2 to about 8 seconds, about 2 to about 6 seconds, about 2 to about 5 seconds, about 2 to about 4 seconds, about 3 to about 10 seconds, about 3 to about 8 seconds, about 3 to about 6 seconds, about 3 to about 5 seconds, about 3 to about 4 seconds, about 1 second, about 2 seconds, and/or about 3 seconds.

Various devices can be used to heat the liquid and aerosolize the supplement. In particular, devices containing an atomizer are used and the atomizer is used to heat the liquid. Exemplary devices contain an atomizer and a power source, such as a battery, connected to the atomizer. In some cases, the atomizer comprises a heating element to vaporize the liquid. In some cases, the atomizer further comprises a delivery mechanism that actively or passively transports the liquid to the heating element. In some cases, the atomizer further comprises a wicking material as a delivery mechanism to draw the liquid to the heating element. In some embodiments, the atomizer does not contain a wicking material. In some embodiments, the liquid is dripped directly onto the heating element. In some embodiments, the atomizer is a ceramic atomizer. In some embodiments, the heating element has a resistance of about 0.4 ohms to about 6 ohms, for example, about 0.8 ohms to about 5 ohms, about 1 ohm to about 4 ohms, about 1 ohm to about 2 ohms, and/or about 2 ohms to about 3 ohms. Suitable voltages for the battery that is connected to the heating element include, but are not limited to, 1- to 9.1-volts, 3- to 6-volts, 4- to 5-volts, 4- to 4.5-volts, and/or 4.15-volts. In some cases, the power generated by the battery that is connected to the heating element is about 0.2 to about 200 Joules/second, for example, about 0.3 to about 100 Joules/second, about 0.4 to about 75 Joules/second, about 0.5 to about 50 Joules/second, about 1 to about 40 Joules/second, about 2 to about 20 Joules/second, and/or about 3 to about 10 Joules/second. Suitable materials from which the wicking material is comprised include, but are not limited to, cotton, silica, polyester, ceramic, fiberglass, and stainless steel.

The vapor produced by the methods described herein contains particles having a particle size suitable for inhalation and absorption in the lungs of a subject. In some cases, the vapor contains particles having a mass mean aerodynamic diameter of less than about 50 microns for example, about 0.1 micron to about 15 microns, about 1 nm to about 500 nm, about 10 nm to about 250 nm, and/or about 100 nm to about 200 nm.

Inhalation by a subject of a vapor prepared by the methods described herein advantageously increases plasma levels of the supplement in the subject. In some cases, plasma levels of the supplement about 1 to about 120 minutes after inhalation of the supplement (e.g., about 30 to about 90 minutes, about 60 to about 90 minutes, about 70 to 80 minutes and/or about 75 minutes after inhalation) are detectably increased compared to plasma levels before inhalation of the vapor, for example, by at least 3% (e.g., at least 3.5%, at least 4%, at least 4.5%, at least 5%, and/or at least 5.5%).

Certain aspects of the disclosure are now explained further via the following non-limiting examples.

### EXAMPLES

### Example 1: Aerosolization Temperature

The temperature of aerosolization is directly related to the power in watts running through the atomizer. Below 20 W, the temperature output of the device is from about 25° C. to about 300° C. with no prior usage.

The power in watts (W) can be obtained from the battery voltage (V) and the heating element resistance (R) based on the relationship W=V²/R. For example, the power output for a system having a 4.2 V battery and a resistance of 3 W is 5.9 W (4.22/3). In another example, the power output for a system having a 3.2 V battery and a resistance of 3 W is 3.4 W (3.22/3). In another example, the power output for a system having a 4.2 V battery and a resistance of 1.8 W is 9.8 W (4.22/1.8).

### Example 2: Vaporization of a Vitamin B12 Formulation

An electronic inhaler device powered by a 4.2V, 320 mAh lithium-ion battery was used to aerosolize a sample containing vitamin B12 in vegetable glycerin and 1,3-propanediol. The sample was heated to between 100 to 260° C. with a 3 ohm heating element in about 2 to about 3 second intervals with about 5 to about 10 second rest periods until a total volume of 100 mg was collected. The mixture contained 60% glycerin and 40% 1,3-propanediol to which 2 mg/ml of vitamin B12 was added. A vacuum was used to draw the vapors through the e-inhaler into an impinger, thereby trapping vaporized vitamin B12 in a water reservoir present in the impinger. Analysis of the water reservoir in the impinger (HPLC) showed vitamin B12 was present. Detection of vitamin B12 in the water reservoir confirms that the vitamin B12 was vaporized.

### Example 3: Vaporization of a Melatonin Formulation

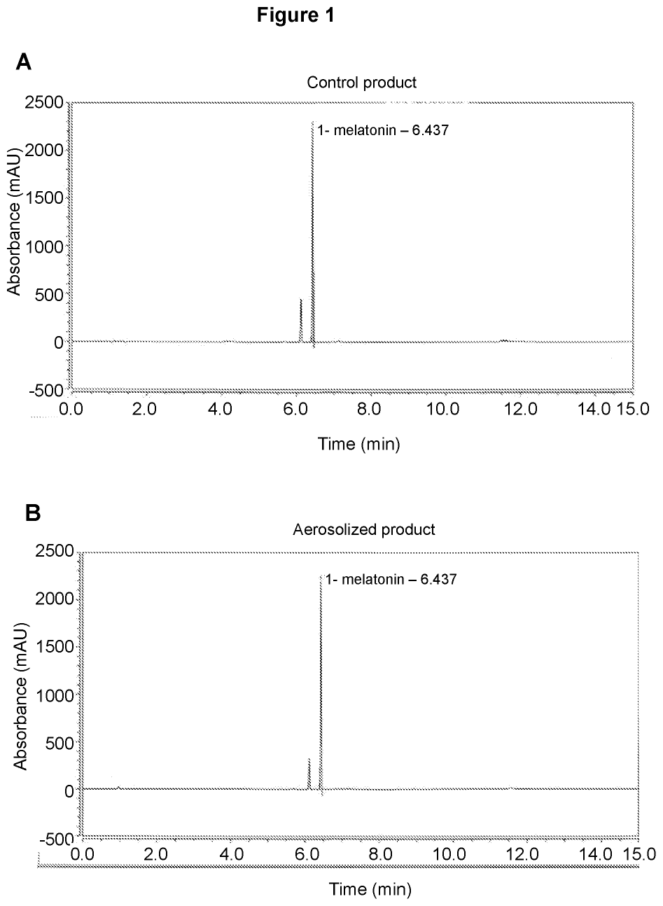

An electronic inhaler device powered by a 4.2V, 320 mAh lithium-ion battery was used to aerosolize a sample containing melatonin in vegetable glycerin and 1,3-propanediol. The sample was heated to between 100 to 260° C. with a 3 ohm heating element in about 2 to about 3 second intervals with about 5 to about 10 second rest periods until a total volume of 100 mg was collected. The mixture contained 60% glycerin and 40% 1,3-propanediol to which 33 mg/ml melatonin was added. A vacuum was used to draw the vapors through the e-inhaler into an impinger, thereby trapping vaporized melatonin in a water reservoir present in the impinger. Analysis of the water reservoir in the impinger (HPLC) showed melatonin was present (FIG. 1(A), B)). Detection of melatonin in the water reservoir confirms that the melatonin was vaporized.

### Example 4: Vaporization of a L-Theanine/Caffeine Formulation

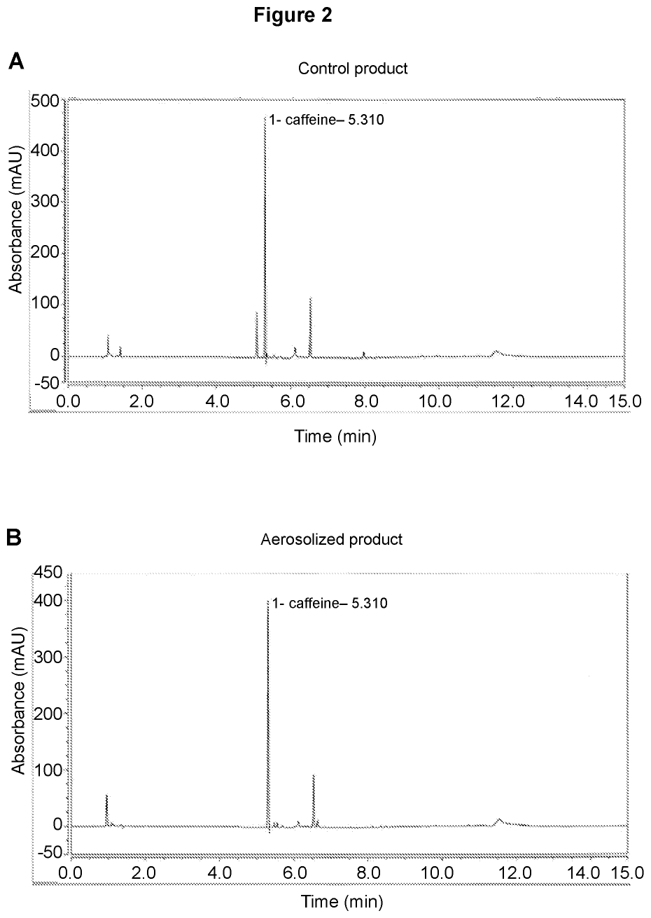

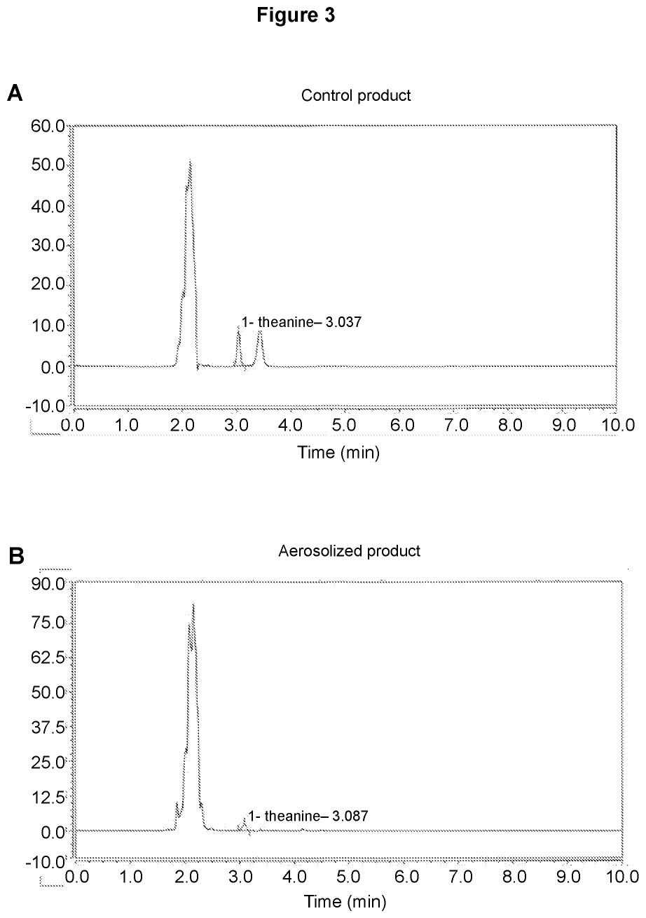

An electronic inhaler device powered by a 4.2V, 320 mAh lithium-ion battery was used to aerosolize a sample containing L-theanine and caffeine in vegetable glycerin and 1,3-propanediol. The sample was heated to between 100 to 260° C. with a 3 ohm heating element in about 2 to about 3 second intervals with about 5 to about 10 second rest periods until a total volume of 100 mg was collected. The mixture contained 60% glycerin and 40% 1,3-propanediol to which 7.7 mg/ml L-theanine, 4.6 mg/ml caffeine, and 2 mg/ml vitamin B12 was added. A vacuum was used to draw the vapors through the e-inhaler into an impinger, thereby trapping vaporized L-theanine and caffeine in a water reservoir present in the impinger. Analysis of the water reservoir in the impinger (HPLC) showed that both L-theanine and caffeine were present (FIG. 2(A), (B)), and demonstrated similar levels of L-theanine and caffeine as the original samples prior to vaporization (FIG. 3 (A), (B)). Detection of L-theanine and caffeine in the water reservoir confirms that both L-theanine and caffeine were vaporized.

### Example 5: Vaporization of a B Vitamin Formulation

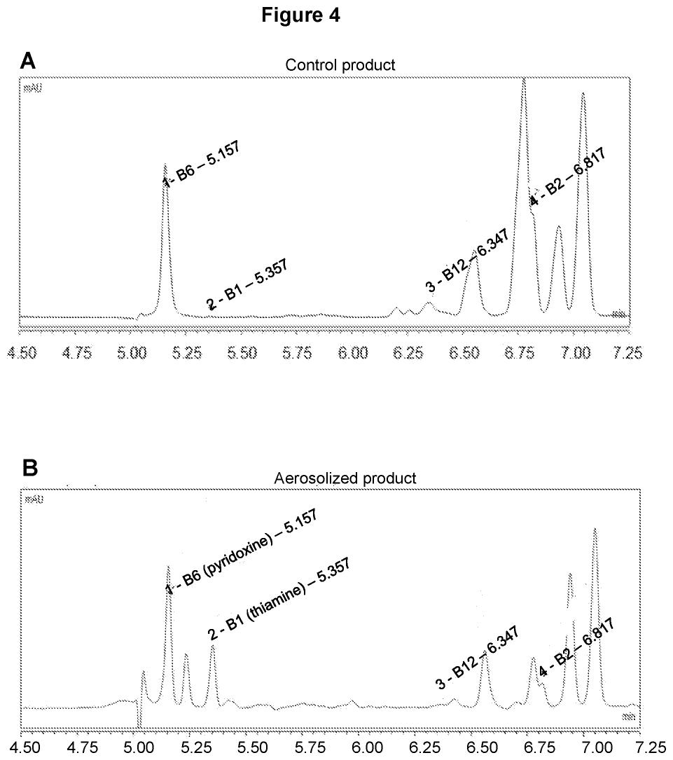

An electronic inhaler device powered by a 4.2V, 320 mAh lithium-ion battery was used to aerosolize a sample containing vitamins B1 (thiamine), B2 (riboflavin), B6 (pyridoxine) and B12 (cobalamin) in vegetable glycerin and 1,3-propanediol. The sample was heated to between 100 to 260° C. with a 3 ohm heating element in about 2 to about 3 second intervals with about 5 to about 10 second rest periods until a total volume of 100 mg was collected. The mixture contained 60% glycerin and 40% 1,3-propanediol to which vitamins B1 (1.3 mg/ml), B2 (1.3 mg/ml), B6 (2.7 mg/ml) and B12 (2.5 mcg/ml) were added. A vacuum was used to draw the vapors through the e-inhaler into an impinger, thereby trapping vaporized vitamins in a water reservoir present in the impinger. Analysis of the water reservoir in the impinger (HPLC) showed vitamins B1, B2, B6 and B12 were present (FIG. 4(B)), and demonstrated similar levels of each of vitamins B1, B2, B6 and B12 in vapor, as compared with the original sample of liquid vitamin formula prior to vaporization (FIG. 4(A)). Detection of each of these vitamins in the water reservoir confirms that vitamins B1, B2, B6 and B12 were vaporized.

### Example 6: Quality Control Studies of Vaporization Products

Following the successful phase shift tests, a Quality Control Analysis was completed to detect chemical components and to eliminate any undesirable substances. Undesirable substances include nicotine, diacetyl, acetoin and pentanedione. The analysis was conducted on the samples utilizing both HPLC and gas chromatography+mass spectroscopy (GCMS). Zero undesirable or volatile substances were detected, as shown in Table 1. Propylene Glycol (PG), another undesirable substance, is also not present or detected in the vaporized compositions (data not shown).

TABLE 1
Quality Control Analysis of Vitamin Vapor.

| Component | Concentration (μg/g) |
|---|---|
| Diacetyl | ND < 2 |
| Pentanedione | ND < 2 |
| Acetoin | ND < 11 |
| Nicotine | ND < 30 |

ND: Not detected

### Example 7: In Vivo Efficacy of a Vaporized Vitamin Formulation

An electronic inhaler device powered by a 4.2V, 320 mAh lithium-ion battery was used to aerosolize a sample containing vitamin B12 in organic vegetable glycerin and 1,3-propanediol and the aerosolized sample was inhaled by test subjects. The sample was heated to between 75 to 260° C. with a 3 ohm heating element in about 2 to about 3 second intervals with about 5 to about 10 second rest periods for a total of about 90 minutes. The mixture contained 60% glycerin and 40% 1,3-propanediol to which 2 mg/ml vitamin B12 was added. Serum vitamin B12 levels were measured in the test subjects before inhalation of the aerosolized sample and 75 minutes after initial inhalation of the aerosolized sample. Before inhalation, serum B12 levels averaged 403.0 pg/ml. After inhalation, serum B12 levels averaged 424.7 pg/ml, a 5.4% increase compared to B12 levels before inhalation of the sample. This study demonstrates that the aerosolized vitamin B12 rapidly entered the bloodstream when inhaled by the test subjects.

### Example 8: Sustained Serum Level Increase with Use of a Vaporized Vitamin Formulation

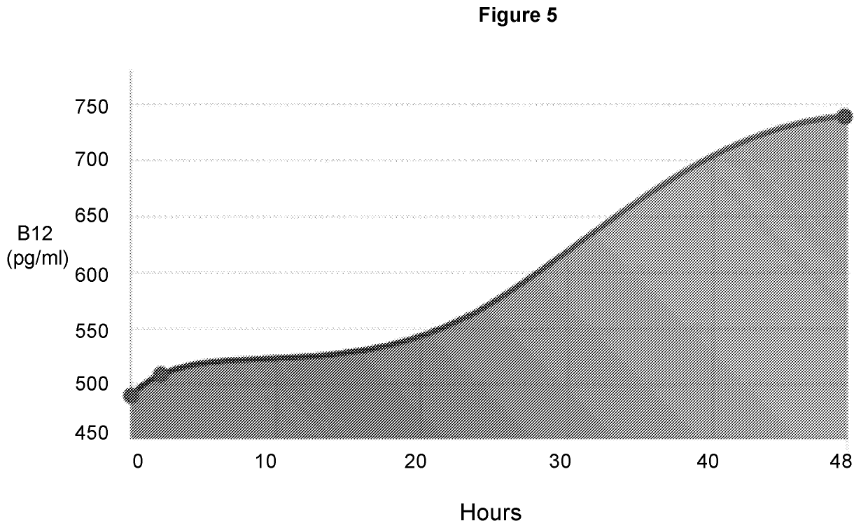

An electronic inhaler device powered by a 4.2V, 320 mAh lithium-ion battery was used to aerosolize a sample containing vitamin B12 in organic vegetable glycerin and 1,3-propanediol and the aerosolized sample was inhaled by one test subject. The sample was heated to between 75 to 260° C. with a 3 ohm heating element in about 2 to about 3 second intervals with about 5 to about 10 second rest periods for a total of about 90 minutes. The mixture contained 60% glycerin and 40% 1,3-propanediol to which 2 mg/ml vitamin B12 was added. Test subject took 60 inhalations/breaths (one dose) of the vitamin B12 product (20 mg/ml) per day for 3 consecutive days. Each of the three doses was administered at 24 hour intervals. Serum vitamin B12 levels were measured in the test subject before inhalation of the aerosolized sample and 2 hours after inhalation of the initial and final dose of the aerosolized sample. Before inhalation, serum B12 levels averaged 496.0 pg/ml (FIG. 5). Two hours after inhalation of the initial dose, serum B12 levels averaged 517.0 pg/ml, and two hours after the final dose, serum B12 levels averaged 728.0 pg/ml (FIG. 5). A human subject is deficient of vitamin B12 when blood serum levels are below 200 pg/ml. Continued use of the vitamin B12 product over a 48 hour period increased B12 blood serum levels by 232 pg/ml. This study demonstrates that the aerosolized vitamin B12 product increases B12 blood levels when inhaled by a test subject, providing rapid absorption of vitamin B12 in a non-invasive manner and which bypasses all of the digestive inefficiencies present in oral administration.

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

1. A method of preparing an inhalable supplement comprising aerosolizing a liquid comprising a carrier and an inhalable supplement by heating the liquid to a temperature of about 25° C. to about 300° C. using an atomizer, thereby providing an inhalable vapor containing the supplement, wherein the liquid is free of nicotine.

2. A method of treating a subject with a supplement comprising administering to the subject a therapeutically effective amount of an inhalable vapor containing the supplement.

3. The method according to claim 2, wherein the inhalable vapor comprises a liquid comprising a carrier and a supplement, and wherein the liquid is aerosolized by heating the liquid to a temperature of about 25° C. to about 300° C. using an atomizer, and wherein the liquid is free of nicotine.

4. The method according to claim 1, wherein the liquid is a mixture.

5. (canceled)

6. The method according to claim 1, wherein the liquid comprises about 51% (w/w) to about 99.99999% (w/w) carrier and/or the liquid comprises about 0.00001% (w/w) to about 49% (w/w) inhalable supplement.

7. The method according to claim 1, wherein the carrier comprises glycerin, 1,2-propanediol, 1,3-propanediol, or combinations thereof.

8. The method according to claim 1, wherein the carrier comprises glycerin and 1,3-propanediol present in a weight ratio of 1:1000 to 1000:1 or glycerin and 1,2-propanediol present in a weight ratio of 1:1000 to 1000:1.

9. The method according to claim 1, wherein the supplement comprises vitamin B1, vitamin B2, vitamin B6, vitamin B12, ascorbic acid, biotin, or combinations thereof.

10. The method according to claim 1, wherein the supplement comprises melatonin.

11. The method according to claim 1, wherein the supplement comprises L-theanine, caffeine, or a combination thereof.

12. The method according to claim 1, wherein the supplement has a boiling point greater than the temperature to which the liquid is heated.

13. The method according to claim 1, wherein the liquid is heated for about 1 to about 10 seconds.

14. The method according to claim 1, wherein the atomizer comprises a heating element to vaporize the liquid.

15. The method according to claim 14, wherein the heating element has a resistance of about 0.4 ohms to about 6 ohms and/or the heating element is connected to a 1- to 9.1-volt battery.

16. The method according to claim 1, wherein the vapor contains particles having a mass mean aerodynamic diameter of less than about 50 microns.

17. The method according to claim 1, wherein inhalation of the vapor by a subject increases plasma levels of the supplement in the subject.

18. The method according to claim 17, wherein plasma levels of the supplement about 60 to about 90 minutes after inhalation of the vapor are increased by at least 3% compared to plasma levels before inhalation of the vapor.

19. The method according to claim 1, wherein the atomizer further comprises a heating element connected to a battery, wherein the power generated by the battery connected to the heating element is about 0.2 to about 200 Joules/second, and wherein the liquid is heated for 1 to 10 seconds.

20. A method according to claim 2, wherein the inhalable vapor is administered in a single dose or in multiple doses.

21. A kit for treating a subject with a supplement comprising:
a. a therapeutically effective amount of an inhalable vapor containing the supplement;
b. an apparatus for administering said compound; and
c. instructions for use.
